(19) United States
(12) Patent Application Publication
(10) Pub. No.: US 2024/0306961 A1
(43) Pub. Date: Sep. 19, 2024

(54) Capillary Blood Collection Device

(71) Applicant: Becton, Dickinson and Company, Franklin Lakes, NJ (US)

(72) Inventors: Alex F. Fricke, Bloomingdale, NJ (US); Charles Peter Althoff, West Nyack, NY (US); Anthony V. Torris, Mercer Island, WA (US); Vlad Yakhnich, Park Ridge, NJ (US)

(21) Appl. No.: 18/572,980

(22) PCT Filed: Jun. 23, 2022
(86) PCT No.: PCT/US22/34630

§ 371 Date: Dec. 21, 2023

Related U.S. Application Data
(60) Provisional application No. 63/216,245, filed on Jun. 29, 2021.

(51) Int. Cl. A61B 5/15; A61B 5/151

(52) U.S. Cl. CPC A61B 5/150022; A61B 5/150343; A61B 5/150786; A61B 5/151

(57) ABSTRACT

A device for obtaining a blood sample may include a holder for receiving a sample source. the holder having an actuation portion and a port: a blood collector attachment removably connected to the holder; and a collection container removably connectable to the blood collector attachment. the container defining a collection cavity, and the collection container comprising at least one fill line provided on a surface of the collection container.

### CROSS-REFERENCE TO RELATED APPLICATION

The present application claims priority to U.S. Provisional Application Ser. No. 63/216,245, filed Jun. 29, 2021, entitled “Capillary Blood Collection Device”, the entire disclosure of which is hereby incorporated by reference in its' entirety.

### BACKGROUND OF THE INVENTION

### Field of the Invention

The present disclosure relates generally to a device for obtaining a biological sample. More particularly, the present disclosure relates to an integrated finger-based capillary blood collection device with the ability to lance and squeeze a finger, collect, stabilize, and dispense a blood sample in a controlled manner.

### Description of Related Art

Devices for obtaining and collecting biological samples, such as blood samples, are commonly used in the medical industry. One type of blood collection that is commonly done in the medial field is capillary blood collection which is often done to collect blood samples for testing. Certain diseases, such as diabetes, require that the patient's blood be tested on a regular basis to monitor, for example, the patient's blood sugar levels. Additionally, test kits, such as cholesterol test kits, often require a blood sample for analysis. The blood collection procedure usually involves pricking a finger or other suitable body part in order to obtain the blood sample. Typically, the amount of blood needed for such tests is relatively small and a small puncture wound or incision normally provides a sufficient amount of blood for these tests. Various types of lancet devices have been developed which are used for puncturing the skin of a patient to obtain a capillary blood sample from the patient.

Many different types of lancet devices are commercially available to hospitals, clinics, doctors' offices, and the like, as well as to individual consumers. Such devices typically include a sharp-pointed member such as a needle, or a sharp-edged member such as a blade, that is used to make a quick puncture wound or incision in the patient's skin in order to provide a small outflow of blood. It is often physiologically and psychologically difficult for many people to prick their own finger with a hand-held needle or blade. As a result, lancet devices have evolved into automatic devices that puncture or cut the skin of the patient upon the actuation of a triggering mechanism. In some devices, the needle or blade is kept in a standby position until it is triggered by the user, who may be a medical professional in charge of drawing blood from the patient, or the patient himself or herself. Upon triggering, the needle or blade punctures or cuts the skin of the patient, for example, on the finger. Often, a spring is incorporated into the device to provide the “automatic” force necessary to puncture or cut the skin of the patient.

One type of contact activated lancet device that features automatic ejection and retraction of the puncturing or cutting element from and into the device is U.S. Pat. No. 9,380,975, which is owned by Becton, Dickinson and Company, the assignee of the present application. This lancet device includes a housing and a lancet structure having a puncturing element. The lancet structure is disposed within the housing and adapted for movement between a retaining or pre-actuated position wherein the puncturing element is retained within the housing, and a puncturing position wherein the puncturing element extends through a forward end of the housing. The lancet device includes a drive spring disposed within the housing for biasing the lancet structure toward the puncturing position, and a retaining hub retaining the lancet structure in the retracted position against the bias of the drive spring. The retaining hub includes a pivotal lever in interference engagement with the lancet structure. An actuator within the housing pivots the lever, thereby moving the lancet structure toward the rearward end of the housing to at least partially compress the drive spring, and releases the lever from interference engagement with the lancet structure. The blood sample that is received is then collected and/or tested. This testing can be done by a Point-of-Care (POC) testing device or it can be collected and sent to a testing facility.

Currently, capillary blood collection workflow is a complex multi-step process requiring high skill level. The multi-step nature of this process introduces several variables that could cause sample quality issues such as hemolysis, inadequate sample stabilization, and micro-clots. The use of lancet devices for obtaining blood samples can result in several variables that effect the collection of the capillary blood sample, including, but not limited to, holding the lancet still during the testing, obtaining sufficient blood flow from the puncture site, adequately collecting the blood, preventing clotting, and the like. Some of the most common sources of process variability are: (1) inadequate lancing site cleaning and first drop removal which can potentially result in a contaminated sample; (2) inconsistent lancing location and depth which could potentially result in insufficient sample volume and a large fraction of interstitial fluid; (3) inconsistent squeezing technique and excessive pressure near the lancing site to promote blood extraction (e.g., blood milking) which could potentially result in a hemolyzed sample; (4) variable transfer interfaces and collection technique which could potentially result in a hemolyzed or contaminated sample; and (5) inadequate sample mixing with an anticoagulant which could potentially result in micro-clots.

Capillary collection blood draws are typically performed by health care workers either using their fingers to manually squeeze the tissue around the puncture site or by a device using vacuum pressure to pull blood from the site.

Manually squeezing the collection site is a highly technique dependent process that leads to very large variation in success rate and sample quality (as measured by hemolysis—blood cell rupture). Health care workers typically adjust the pressure and rate at which they squeeze to compensate for patient-dependent differences in blood flow. Squeezing harder helps blood flow more quickly but also increases hemolysis. The location of squeezing also varies between health care workers depending on personal preference, experience, and hand fatigue. Some workers may even perform a process called “milking” of fingers, where they apply pressure starting at the base of the finger and slide towards the tip of finger. This process is discouraged as leading to poor sample quality by domestic and international health organizations.

Vacuum-powered devices standardize the pressure and technique of blood flow, but are typically plagued by poor overall blood flow. The maximum pressure than can be applied is limited by the difference between atmospheric pressure and absolute vacuum (˜14 psi), and devices only operate at a fraction of absolute vacuum. For reference, grip strength of men and women range from 50-100 lbs. on average, illustrating why manual methods are instead affected by hemolysis rather than flow. Vacuum methods also apply consistent pressure, limiting the ability of the tissue to replenish with blood.

Thus, there is a need in the art for a device that has the ability to lance and squeeze the finger, collect the sample, stabilize the sample, and subsequently dispense the sample in a controlled manner. There is also a need in the art for a device that simplifies and streamlines the capillary blood collection by eliminating workflow variabilities which are typically associated with low sample quality including hemolysis and micro-clots. There is still a further need in the art for a closed system collection and transfer that eliminate blood exposure and device reuse. There is still a further need in the art for a device that: (1) introduces flexibility in the accommodation of different capillary blood collection and transfer container; (2) has the capability to generate high quality uniformly mixed/stabilized capillary blood samples; (3) has the capability to generate on-board plasma from capillary plasma samples; (4) has the capability to collect large capillary blood samples (>50-500 μL) at reduced pain; (5) contains a unique sample identifier that is paired with patient information at the time of collection; (6) has the capability to collect capillary blood and perform on-board diagnostics; and (7) has multiple collection ports to collect a blood sample into different containers having the same or different anticoagulants. There is a further need in the art for a capillary blood collection device that includes a standardized and controlled location of applied pressure, an applied pressure that is high enough for adequate blood flow but below hemolysis thresholds, a defined rhythmic application of pressure rather than consistent pressure to allow blood to replenish in the finger, increasing average blood flow rate, and a reduced user fatigue by lowering maximum applied force by the operator.

### SUMMARY OF THE INVENTION

The present disclosure is directed to a device for obtaining a biological sample, such as a capillary blood collection device, which meets the needs set forth above and has the ability to lance and squeeze the finger, collect the sample, stabilize the sample, and subsequently dispense the sample in a controlled manner. The device also simplifies and streamlines the capillary blood collection by eliminating workflow variabilities which are typically associated with low sample quality including hemolysis and micro-clots.

The present disclosure includes a self-contained and fully integrated finger-based capillary blood collection device with ability to lance, collect, and stabilize high volume capillary blood sample, e.g., up to or above 500 microliters. The device simplifies and streamlines high volume capillary blood collection by eliminating workflow steps and variabilities which are typically associated with low sample quality including hemolysis, micro-clots, and patient discomfort. The device comprises a retractable lancing mechanism that can lance the finger and an associated blood flow path which ensures attachment and transfer of the capillary blood from the pricked finger site to the collection container. The device also includes a holder that can be cyclically squeezed to stimulate, i.e., pump, blood flow out of the finger and also an anticoagulant deposited in the flow path or collection container to stabilize collected sample.

According to one design, the device can comprise discrete components such as a holder, a lancet, and a collection container. According to another design, the lancet and collection container can be integrated into one device which is then used with the holder. According to yet another design, the holder, lancet, and collection container can be integrated into a single system. Any of these designs are envisioned to be used as a self-standing disposable device and/or in association with an external power source for pain reduction control. The capillary blood collection device can serve as a platform for various capillary blood collection containers ranging from small tubes to capillary dispensers, as well as on-board plasma separation modules. This capability extends the product flexibility to various applications including dispensing to a Point-of-Care (POC) cartridge or to a small collection tube transfer which can be used in a centrifuge or an analytical instrument.

In one embodiment of the present disclosure, a device for obtaining a blood sample may include a holder for receiving a sample source, the holder having an actuation portion and a port; a blood collector attachment removably connected to the holder; and a collection container removably connectable to the blood collector attachment, the container defining a collection cavity, and the collection container comprising at least one fill line provided on a surface of the collection container.

In one embodiment of the present disclosure, the at least one fill line may be configured to indicate when a predetermined volume of the blood sample has been filled in the collection container. The collection container may include two fill lines provided on the surface of the collection container. The at least one fill line of the collection container may be color coded. The at least one fill line of the collection container may have a lighter shade of color. The at least one fill line of the collection container may have a darker shade of color.

In one embodiment of the present disclosure, a device for obtaining a blood sample may include a holder for receiving a sample source, the holder having an actuation portion and a port; and a blood collector attachment removably connected to the holder, wherein the blood collector attachment includes a flash chamber to indicate when the blood sample has been drawn from a patient's finger held in the holder.

In one embodiment of the present disclosure, the blood collector attachment may be made of a material that is transparent such that a user of the device can visually identify the blood sample flowing through the blood collector attachment. The blood collector attachment may include a light rib on an inner surface thereof to visually indicate when the blood sample is moving through the blood collector attachment.

In one embodiment of the present disclosure, a device for obtaining a blood sample may include a holder for receiving a sample source, the holder having an actuation portion and a port; and a lancet removably connected to the holder, wherein the lancet is positioned relative to the holder so as to obstruct a patient's view of the lancet removably connected to the holder.

In one embodiment of the present disclosure, the port of the holder may be configured to obstruct the patient's view of the lancet. Upon connection of the lancet into the holder, an audible clicking sound may be generated to indicate that a positive connection between the lancet and the holder has been established.

In one embodiment of the present disclosure, a device for obtaining a blood sample may include a holder for receiving a sample source, the holder having an actuation portion and a port; and a blood collector attachment removably connected to the holder; and a collection container removably connected to the blood collector attachment, wherein a label positioned on the collection container is oriented so as to obstruct a patient's view of an inner cavity of the collection container.

In one embodiment of the present disclosure, the label may be positioned on the collection container at a location on the collection container underneath the holder. The label may cover a portion of the collection container so the patient is unable to view the blood sample being directed into the collection container.

The present invention is also disclosed in the following clauses:

Clause 1: A device for obtaining a blood sample, the device comprising: a holder for receiving a sample source, the holder having an actuation portion and a port; a blood collector attachment removably connected to the holder; and a collection container removably connectable to the blood collector attachment, the container defining a collection cavity, and the collection container comprising at least one fill line provided on a surface of the collection container.

Clause 2: The device of Clause 1, wherein the at least one fill line is configured to indicate when a predetermined volume of the blood sample has been filled in the collection container.

Clause 3: The device of Clause 1 or 2, wherein the collection container comprises two fill lines provided on the surface of the collection container.

Clause 4: The device of any of Clauses 1-3, wherein the at least one fill line of the collection container is color coded.

Clause 5: The device of any of Clauses 1-4, wherein the at least one fill line of the collection container has a lighter shade of color.

Clause 6: The device of Clause 5, wherein the at least one fill line of the collection container has a darker shade of color.

Clause 7: A device for obtaining a blood sample, the device comprising: a holder for receiving a sample source, the holder having an actuation portion and a port; and a blood collector attachment removably connected to the holder, wherein the blood collector attachment includes a flash chamber to indicate when the blood sample has been drawn from a patient's finger held in the holder.

Clause 8: The device of Clause 7, wherein the blood collector attachment is made of a material that is transparent such that a user of the device can visually identify the blood sample flowing through the blood collector attachment.

Clause 9: The device of Clause 7 or 8, wherein the blood collector attachment includes a light rib on an inner surface thereof to visually indicate when the blood sample is moving through the blood collector attachment.

Clause 10: A device for obtaining a blood sample, the device comprising: a holder for receiving a sample source, the holder having an actuation portion and a port; and a lancet removably connected to the holder, wherein the lancet is positioned relative to the holder so as to obstruct a patient's view of the lancet removably connected to the holder.

Clause 11: The device of Clause 10, wherein the port of the holder is configured to obstruct the patient's view of the lancet.

Clause 12: The device of Clause 10 or 11, wherein, upon connection of the lancet into the holder, an audible clicking sound is generated to indicate that a positive connection between the lancet and the holder has been established.

Clause 13: A device for obtaining a blood sample, the device comprising: a holder for receiving a sample source, the holder having an actuation portion and a port; and a blood collector attachment removably connected to the holder; and a collection container removably connected to the blood collector attachment, wherein a label positioned on the collection container is oriented so as to obstruct a patient's view of an inner cavity of the collection container.

Clause 14: The device of Clause 13, wherein the label is positioned on the collection container at a location on the collection container underneath the holder.

Clause 15: The device of Clause 13 or 14, wherein the label covers a portion of the collection container so the patient is unable to view the blood sample being directed into the collection container.

### BRIEF DESCRIPTION OF THE DRAWINGS

FIG. 1 is a perspective view of a holder in accordance with an embodiment of the present invention.

FIG. 2A is a cross-sectional view of a device for obtaining a blood sample from a patient's finger and a lancet in accordance with another embodiment of the present disclosure.

FIG. 2B is a perspective view of a device for obtaining a blood sample from a patient's finger and a sample collection container in accordance with another embodiment of the present disclosure.

FIG. 3 is a perspective view of the device and lancet of FIG. 2A.

FIG. 4 is a side view of the holder of FIG. 1 and a collection container according to an embodiment of the present disclosure.

FIG. 5 is a perspective view of a holder, a blood collector attachment, and a collection container according to an embodiment of the present disclosure.

FIG. 6 is a side view of the holder, the blood collector attachment, and the collection container of FIG. 5.

FIG. 7 is a front view of the holder, the blood collector attachment, and the collection container of FIG. 5.

FIG. 8 is an isolated view of the blood collector attachment of FIG. 5 according to an embodiment of the present disclosure.

FIG. 9 is an isolated view of the blood collector attachment of FIG. 5 according to an embodiment of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”. “vertical”, “horizontal”, “top”, “bottom”, “lateral”, “longitudinal”, and derivatives thereof shall relate to the invention as it is oriented in the drawing figures. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

The present disclosure is directed to a device for obtaining a biological sample, such as a capillary blood collection device, which meets the needs set forth above and has the ability to lance and squeeze the finger, collect the sample, stabilize the sample, and subsequently dispense the sample in a controlled manner. The device also simplifies and streamlines the capillary blood collection by eliminating workflow variabilities which are typically associated with low sample quality including hemolysis and micro-clots.

Blood collection is fundamentally driven by pressure-driven flow. Devices or techniques either reduce the pressure outside the blood vessel (vacuum-powered flow) or increase the pressure inside the vessels. Both approaches increase the difference between the blood vessel pressure and external pressure, and increase the flow rate from inside the vessel to outside where the collection container is present. The location of squeezing can also be critical, as soft tissues (e.g. fat, skin, and musculature) are perfused with blood while hard tissues and joints are poorly perfused or are too mechanically stable to compress without patient pain.

Red blood cells (RBCs) are subject to hemolysis during collection. Hemolysis (RBC destruction) contaminates samples for diagnostic analysis, both by spilling cell contents into the liquid serum of the sample and by coloring the serum red via hemoglobin and interfering with colorimetric reactions. The amount of hemolysis during collection is driven by shear-mediated destruction of the cells due to flow rate and flow path as well as pressure-driven hemolysis where physical compression of tissues and vessels can damage cells. Hemolysis can therefore be controlled by ensuring that applied pressures and flows are not too high in any of the locations of the finger being squeezed.

The present disclosure includes a self-contained and fully integrated finger-based capillary blood collection device with ability to lance, collect, and stabilize high volume capillary blood sample, e.g., up to or above 500 microliters. The device simplifies and streamlines high volume capillary blood collection by eliminating workflow steps and variabilities which are typically associated with low sample quality including hemolysis, micro-clots, and patient discomfort. The device comprises a retractable lancing mechanism that can lance the finger and an associated blood flow path which ensures attachment and transfer of the capillary blood from the pricked finger site to the collection container. The device also includes a holder that can be cyclically squeezed to stimulate, i.e., pump, blood flow out of the finger and also an anticoagulant deposited in the flow path or collection container to stabilize collected sample.

According to one design, the device can comprise discrete components such as a holder, a lancet, and a collection container. According to another design, the lancet and collection container can be integrated into one device which is then used with the holder. According to yet another design, the holder, lancet, and collection container can be integrated into a single system. Any of these designs are envisioned to be used as a self-standing disposable device and/or in association with an external power source for pain reduction control. The capillary blood collection device can serve as a platform for various capillary blood collection containers ranging from small tubes to capillary dispensers, as well as on-board plasma separation modules. This capability extends the product flexibility to various applications including dispensing to a Point-of-Care (POC) cartridge or to a small collection tube transfer which can be used in a centrifuge or an analytical instrument.

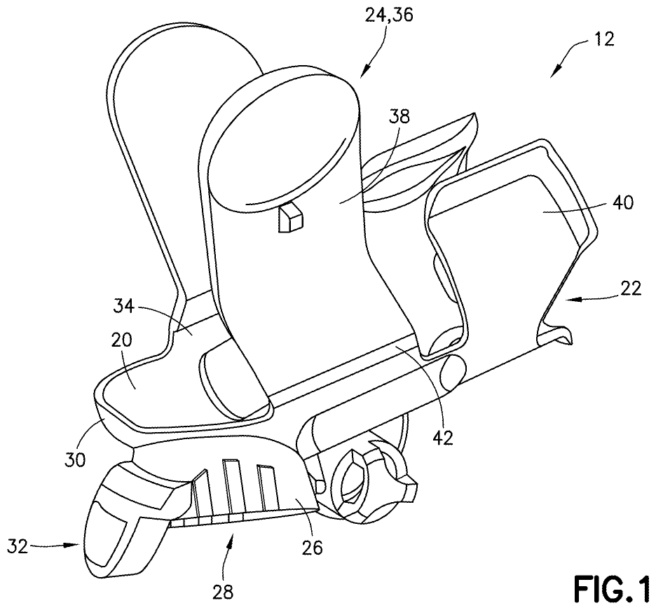

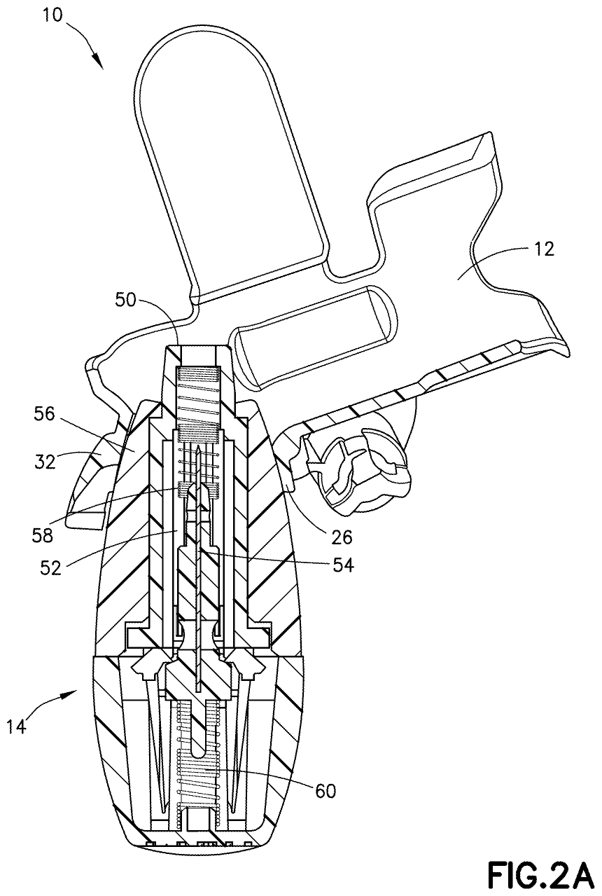

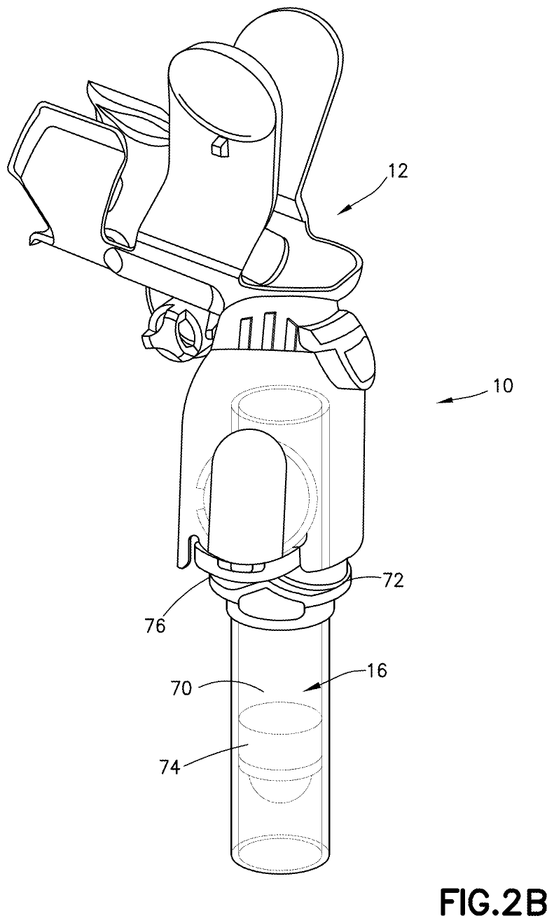

Referring to FIGS. 1 and 2A, in an exemplary embodiment, a device 10 of the present disclosure includes discrete components, e.g., a holder 12 (as shown in FIG. 1), a lancet housing or lancet 14 (as shown in FIG. 2A), and a collection container 16. In another exemplary embodiment, a semi-integrated device of the present disclosure may include an at-angle flow and include an integrated lancet housing and collection container which can be connected with a separate holder. In another exemplary embodiment, a semi-integrated device of the present disclosure may have an in-line flow and include an integrated lancet housing and collection container which can be connected with a separate holder. In another exemplary embodiment, an integrated device of the present disclosure may have an at-angle flow and include an integrated holder, lancet housing, and collection container. In another exemplary embodiment, an integrated device of the present disclosure may have an in-line flow and include an integrated holder, lancet housing, and collection container.

Referring to FIG. 1, an exemplary embodiment of a holder 12 of the present disclosure that is able to receive a sample source, e.g., a finger 19, for supplying a biological sample, such as a blood sample 18, is shown and described. A holder 12 of the present disclosure generally includes a finger receiving portion 20 having a first opening 22 (FIG. 1), an actuation portion 24, a port 26 having a second opening 28, and a finger end guard 30. In one embodiment, the finger end guard 30 provides a stop portion for properly aligning and securing a finger 19 within the holder 12. The finger end guard 30 further assists in ensuring the patient's finger 19 is placed at a proper position within the finger receiving portion 20 so that applied pressure to the patient's finger 19 will result in adequate blood flow.

The first opening 22 of the finger receiving portion 20 is configured for receiving a sample source, e.g., a finger 19, for supplying a biological sample, such as a blood sample 18. It can be appreciated that the sample source could include other parts of the body capable of fitting within the first opening 22. The port 26 is in communication with the finger receiving portion 20. For example, with a finger 19 received within the holder 12, the port 26 is in communication with a portion of the finger 19. A holder 12 of the present disclosure can be sized to accommodate all finger sizes.

The second opening 28 of the port 26 is configured for receiving a lancet housing 14 and a collection container 16 as described in more detail below. In one embodiment, the port 26 includes a locking portion 32 for securely receiving the lancet housing 14 and the collection container 16 within the port 26.

In one embodiment, the actuation portion 24 is transitionable between a first position in which the holder 12 defines a first diameter and a second position which the holder 12 defines a second diameter, wherein the second diameter is less than the first diameter. In one embodiment, the actuation portion 24 is transitionable between a first position in which the holder 12 defines a first elliptical shape, and a second position in which the holder 12 defines a second elliptical shape, wherein the first elliptical shape is different than the second elliptical shape. In this manner, with the holder 12 in the second position with a reduced diameter, a portion of the holder 12 contacts the sample source and the actuation portion 24 of the holder 12 is able to pump and/or extract blood 18 as described in more detail below.

Referring to FIG. 1, in one embodiment, the actuation portion 24 includes a contact member 34. With the actuation portion 24 in the first position, the contact member 34 is in a disengaged position, i.e., the contact member 34 is provided in a first position with respect to a sample source, e.g., the finger 19, such that the contact member 34 may be in slight contact therewith. With the actuation portion 24 in the second position, the contact member 34 is in an engaged position, i.e., the contact member 34 is provided in a second position with respect to the sample source, e.g., the finger 19, such that the contact member 34 is in an applied pressure contact with the finger 19, and the actuation portion 24 of the holder 12 is able to pump and/or extract blood 18. For example, with the contact member 34 in the engaged position, the contact member 34 exerts a pressure on the sample source.

Referring to FIG. 1, in one embodiment, the actuation portion 24 includes a pumping member 36 for applying pressure to the sample source, e.g., the finger 19. In one embodiment, the pumping member 36 comprises a pair of opposed tabs or wings 38. In such an embodiment, each tab 38 may include a contact member 34. In one embodiment, the holder 12 includes a living hinge portion 42. The living hinge portion 42 allows a user to squeeze the wings 38 between a first position (passive state) and a second position (active state). The use of the tabs or wings 38 to draw blood 18 out of a patient's finger 19 minimizes hemolysis while maintaining an adequate flow of blood from the patient's finger 19. A resting position and hinge of the wings 38 are designed to maintain contact and retention with the smallest patient finger that can fit into a holder 12 while flexing to accommodate the largest patient finger within a holder 12 without blood occlusion.

Advantageously, the holder 12 of the present disclosure allows a user to repeatedly squeeze and release the wings 38 to pump and/or extract blood 18 from a finger 19 until a desired amount of blood 18 is filled in a collection container 16. The wings 38 are configured to flex to maintain gentle contact with a range of patient finger sizes that may be used with the holder 12 and to retain the holder 12 on the patient's finger 19.

Advantageously, with the holder 12 placed onto a finger 19, the holder 12 does not constrict the blood flow and defines lancing and finger squeezing locations. The squeezing tabs or wings 38 provide a pre-defined range of squeezing pressure that is consistently applied throughout a finger 19. By doing so, the holder 12 provides a gentle controlled finger massage that stimulates blood extraction and minimizes any potential hemolysis.

Referring to FIG. 1, in one embodiment, the holder 12 includes a stability extension portion 40. This provides additional support for the holder 12 to be securely placed onto a finger 19. In one embodiment, the finger receiving portion 20 forms a generally C-shaped member and includes a plurality of inner gripping members for providing additional grip and support for the holder 12 to be securely placed onto a finger 19. The stability extension portion 40 assists in maintaining contact with the patient's finger 19 during use of the holder 12 while avoiding the blood supply and knuckles of the patient's finger 19.

In one embodiment, the finger receiving portion 20 is formed of a flexible material. In some embodiments, the finger receiving portion 20 and the port 26 are formed from a flexible material.

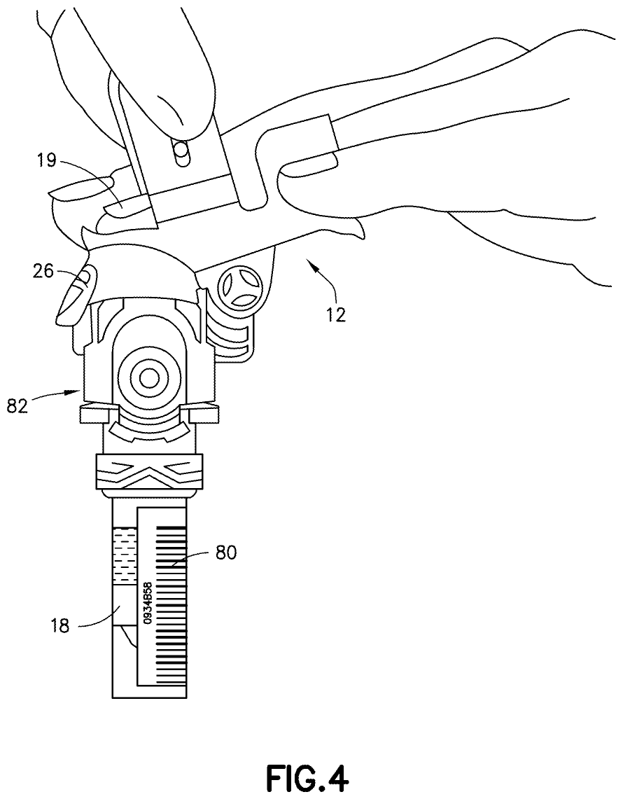

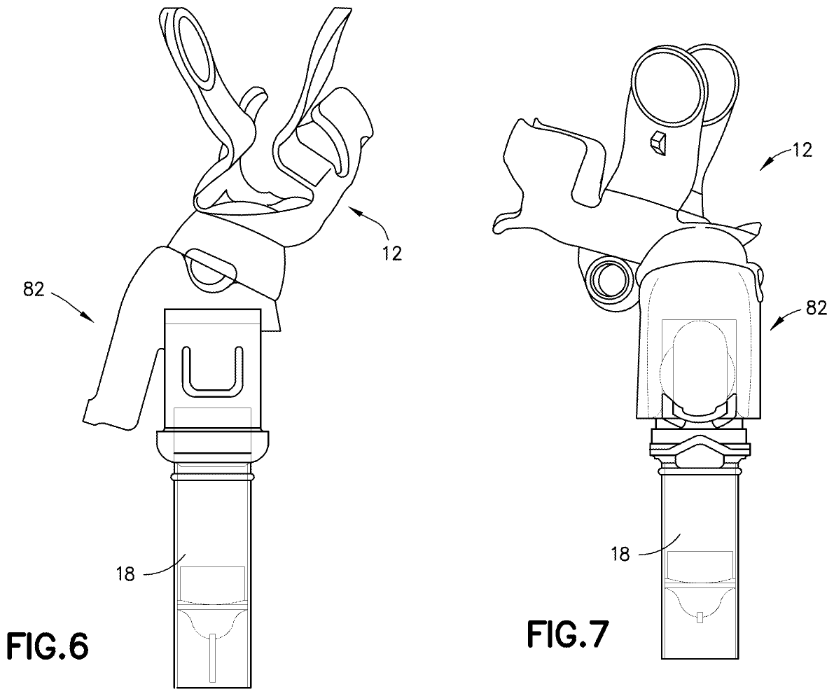

A device 10 for obtaining a blood sample 18 (shown in FIGS. 4-7) of the present disclosure includes a lancet housing or lancet 14 that is removably connectable to a port 26 of a holder 12. Referring to FIG. 2A, in one embodiment, the lancet housing 14 includes an inlet or opening 50, an interior 52, a puncturing element 54, an engagement portion 56, a retractable mechanism 58, and a drive spring 60. In one embodiment, the puncturing element 54 is moveable between a pre-actuated position wherein the puncturing element 54 is retained within the interior 52 of the lancet housing 14 and a puncturing position wherein at least a portion of the puncturing element 54 extends through the inlet 50 of the lancet housing 14 to lance a portion of a finger 19.

In one embodiment, the lancet 14 of the present disclosure is a contact activated lancet and may be constructed in accordance with the features disclosed in U.S. Patent Application Publication No. 2006/0052809 filed May 6, 2005, entitled “Contact Activated Lancet Device”, and commonly assigned with the present application, the entire disclosure of which is hereby expressly incorporated herein by reference thereto.

In one embodiment, the lancet housing 14 may be a separate component from the holder 12 and the collection container 16. In some embodiments, the collection container 16 and the lancet housing 14 form a single component that is removably connectable to the port 26 of the holder 12. In some embodiments, the collection container 16, the lancet housing 14, and the holder 12 form a single component.

Referring to FIG. 2A, in one embodiment, with the holder 12 and the lancet housing 14 being separate components, the lancet housing 14 is removably connectable to the port 26 of the holder 12. In such an embodiment, the lancet housing 14 includes an engagement portion 56. Referring to FIG. 2A, in one embodiment, the lancet housing 14 is pushed into the port 26 of the holder 12 such that the engagement portion 56 of the lancet housing 14 is locked within the locking portion 32 of the holder 12. In this manner, the lancet housing 14 is securely connected and locked to the holder 12 such that the puncturing element 54 of the lancet housing 14 can be activated to lance or puncture a sample source, e.g., a finger 19. In some embodiments, the port 26 of the holder 12 includes a plurality of ribs for securing and locking the lancet 14 or the collection container 16 in the port 26.

To activate the lancet 14, the lancet 14 is pushed against a finger 19 to activate a retractable mechanism 58 of the lancet 14 to lance a finger 19. The lancet 14 of the present disclosure consistently delivers correct lancing depth and a pre-defined lancing location, thus ensuring a sufficient sample volume.

In one embodiment, the lancet 14 includes a drive spring 60 disposed within the interior 52 of the lancet housing 14 for biasing the puncturing element 54 toward the puncturing position. After puncturing, the puncturing element 54 is immediately retracted and safely secured within the interior 52 of the lancet housing 14.

In one embodiment, the lancet 14 of the present disclosure is used to lance the skin of a finger 19 and then a blood sample 18 is squeezed into a collection container 16 as described in more detail below.

In one embodiment, the lancet housing 14 of the present disclosure is used to lance the skin of a finger 19 along a lance path and then a blood sample 18 flows down a blood flow path at an angle to the lance path as described in more detail below.

In one embodiment, the lancet 14 can include a hollow needle. In such an embodiment, the lancet housing 14 of the present disclosure is used to lance the skin of a finger 19 along a lance path and then a blood sample 18 flows along a parallel blood flow path through the hollow needle.

As shown in FIG. 2B, a device 10 for obtaining a blood sample 18 (shown in FIGS. 4-6) of the present disclosure includes a collection container 16 that is removably connectable to the port 26 of the holder 12. The collection container 16 defines a collection cavity 70 for receiving a blood sample 18, a container engagement portion 72, a blood collector portion 74, and a cap or septum 76. Once a desired amount of blood 18 is collected within the container 16, a blood collector portion 74 is detached from the collection device 10 in order to send a collected sample 18 to a diagnostic instrument and/or testing device. The blood collector portion 74 is sealed via the cap or septum 76 once removed from the collection device 10 to protectively seal the blood sample 18 within the collection cavity 70.

In one embodiment, the collection container 16 may be a separate component from the holder 12 and the lancet housing 14. In some embodiments, the collection container 16 and the lancet housing 14 form a single component that is removably connectable to the port 26 of the holder 12. In some embodiments, the collection container 16, the lancet housing 14, and the holder 12 form a single component.

In one embodiment, with the holder 12 and the collection container 16 being separate components, the container 16 is removably connectable to the port 26 of the holder 12. In such an embodiment, the container 16 includes a container engagement portion 72. In one embodiment, the container 16 is pushed into the port 26 of the holder 12 such that the container engagement portion 72 of the container 16 is locked within the locking portion 32 of the holder 12. In this manner, the container 16 is securely connected and locked to the holder 12 such that a blood sample 18 can safely flow from the finger 19 within the holder 12 to the collection cavity 70 of the container 16.

It can be appreciated that several types of collection containers 16 can be used with the device 10 of the present disclosure. It can also be appreciated that the collection container 16 can be associated with a separate dispensing unit or the collection container 16 can include an integral dispensing portion for dispensing the blood 18 to a testing device.

Referring to FIG. 1, use of a device 10 of the present disclosure having discrete components, e.g., a holder 12, a lancet housing or lancet 14, and a collection container 16, will now be described.

Referring to FIG. 1, first a desired finger 19 is cleaned and a holder 12 having an appropriate size for the desired finger 19 is selected and placed onto the finger 19 securely. Next, referring to FIG. 2A, a lancet housing 14 is connected to the port 26 of the holder 12. As discussed above, the lancet housing 14 is pushed into the port 26 of the holder 12 such that the engagement portion 56 of the lancet housing 14 is locked within the locking portion 32 of the holder 12. In this manner, the lancet housing 14 is securely connected and locked to the holder 12 such that the puncturing element 54 (FIG. 2A) of the lancet housing 14 can be activated to lance or puncture a sample source, e.g., a finger 19. With the lancet 14 connected to the port 26 of the holder 12, the lancet 14 is in communication with the finger 19.

When it is desired to activate the lancet 14 to lance the skin of a finger 19, the lancet 14 is pushed against a finger 19 to activate a retractable mechanism 58 (FIG. 2A) of the lancet 14 to lance a finger 19. The lancet 14 of the present disclosure consistently delivers correct lancing depth and a pre-defined lancing location, thus ensuring a sufficient sample volume.

After the finger 19 is lanced to create blood 18 flow from the finger 19, the lancet 14 is removed from the holder 12 and the collection container 16 is pushed into the port 26 of the holder 12. Referring to FIG. 2B, the container 16 is pushed into the port 26 of the holder 12 such that the container engagement portion 72 of the container 16 is locked within the locking portion 32 of the holder 12. In this manner, the container 16 is securely connected and locked to the holder 12 such that a blood sample 18 can safely flow from the finger 19 within the holder 12 to the collection cavity 70 of the container 16.

Referring to FIG. 1, with the container 16 properly secured to the holder 12 for collection of a blood sample 18, a user is able to repeatedly squeeze and release the wings 38 of the holder 12 to pump and/or extract blood 18 from a finger 19 until a desired amount of blood 18 is filled in a collection container 16. Advantageously, with the holder 12 placed onto a finger 19, the holder 12 does not constrict the blood flow and defines lancing and finger squeezing locations. The squeezing tabs or wings 38 provide a pre-defined range of squeezing pressure that is consistently applied throughout a finger 19. By doing so, the holder 12 provides a gentle controlled finger 19 massage that stimulates blood extraction and minimizes any potential hemolysis.

For example, referring to FIG. 1, in one embodiment, the actuation portion 24 includes a contact member 34. With the actuation portion 24 in the first position, the contact member 34 is in a disengaged position, i.e., the contact member 34 is in the first position with respect to the sample source, e.g., the finger 19. With the actuation portion 24 in the second position, the contact member 34 is in an engaged position, i.e., the contact member 34 is in the second position and in applied pressure contact with a sample source, e.g., the finger 19, and the actuation portion 24 of the holder 12 is able to pump and/or extract blood 18. For example, with the contact member 34 in the engaged position, the contact member 34 exerts a pressure on the sample source.

Once a desired amount of blood 18 is collected within the container 16, a blood collector portion 74 is detached from the collection device 10 in order to send a collected sample 18 to a diagnostic instrument and/or testing device. The blood collector portion 74 is scaled via the cap or septum 76 once removed from the collection device 10 to protectively seal the blood sample 18 within the collection cavity 70.

The devices of the present disclosure are compatible with any known testing device, whether the testing device is off-site or a point-of-care testing device. Various point-of-care testing devices are known in the art. Such point-of-care testing devices include test strips, glass slides, diagnostic cartridges, or other testing devices for testing and analysis. Test strips, glass slides, and diagnostic cartridges are point-of-care testing devices that receive a blood sample and test that blood for one or more physiological and biochemical states. There are many point-of-care devices that use cartridge based architecture to analyze very small amounts of blood bedside without the need to send the sample to a lab for analysis. This saves time in getting results over the long run, but creates a different set of challenges versus the highly routine lab environment. Examples of such testing cartridges include the i-STAT® testing cartridge from the Abbot group of companies. Testing cartridges such as the i-STAT® cartridges may be used to test for a variety of conditions including the presence of chemicals and electrolytes, hematology, blood gas concentrations, coagulation, or cardiac markers. The results of tests using such cartridges are quickly provided to the clinician.

The collection container 16 may also contain a sample stabilizer, e.g., an anticoagulant, to stabilize a blood sample 18 and/or a component of a blood sample 18 disposed therein. The collection container 16 may also include at least one fill line(s) corresponding to a predetermined volume of sample. The collection container may also indicate/meter a collected volume of blood.

Any of the devices for obtaining a blood sample of the present disclosure can be used as a self-standing disposable device and/or in association with an external power source for pain reduction control. For example, a portion of holder 12 may include embedded electrodes which receive a signal from an external pain control module to deliver at least one of heat, vibration, or transcutaneous electrical nerve stimulation (TENS) for pain reduction control. The devices for obtaining a blood sample of the present disclosure may also include various options for on-board plasma separation. The devices for obtaining a blood sample of the present disclosure may also include a unique sample identifier that can be paired with patient information at the time of collection. The devices for obtaining a blood sample of the present disclosure may also include on-board diagnostic feedback at the time of collection. A device for obtaining a blood sample of the present disclosure may also allow for dual collection, e.g., the collection of two samples into two separate containers, using multiple collection ports which enable the collection of multiple samples from the same source and treating the samples with different sample stabilizers, such as anticoagulants.

A device for obtaining a blood sample of the present disclosure significantly simplifies and de-skills large volume capillary collection from a finger relative to the conventional capillary collection using lancet and capillary tube. The devices of the present disclosure eliminate blood exposure and prevents device reuse.

The devices for obtaining a blood sample of the present disclosure simplify, deskill, and streamline the collection process. This is all achieved by a self-contained closed system device which after it is placed onto a finger will provide lancing, blood extraction, stabilization, and containment functions, all in one unit.

The devices for obtaining a blood sample of the present disclosure may be associated with a self-standing unit that provides automated pumping, controlled finger squeezing, and automated sample labeling and processing.

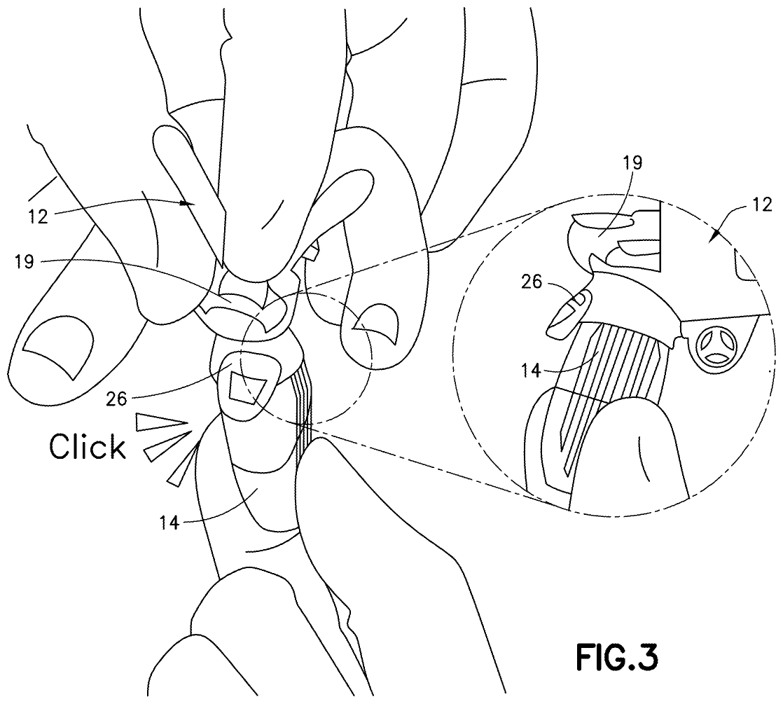

With reference to FIG. 3, according to one embodiment of the present disclosure, as the lancet 14 is connected to the holder 12, an audible “clicking” sound may be generated to notify the user that a secure connection between the lancet 14 and the holder 12 has been achieved. The orientation of the port 26 may be positioned such that the patient's view of the lancet 14 is obscured, resulting in a less stressful interaction for the patient as the patient's finger 19 is pierced by the lancet 14. In one embodiment, the body of the port 26 is positioned above the lancet 14 when the lancet 14 is inserted into the holder 12 such that a portion of the holder 12 also obscures the view of the lancet 14 and the puncture wound site for the patient. In another embodiment, the port 26 may be made of a translucent material so the user can observe the lancet 14 relative to the patient's finger 19 and the flow of blood from the patient's finger 19 after the lancet 14 has pierced the patient's finger 19. This arrangement allows the user to ensure that an adequate piercing of the patient's finger 19 has been achieved by the lancet 14.

With reference to FIG. 4, according to one embodiment of the present disclosure, a label 80 removably attached to the collection container 16 may be oriented on the collection container 16 to obstruct the patient's view of the inner cavity of the collection container 16. After the collection container 16 has been connected to the holder 12 and the patient's finger 19 is pumped, the blood sample 18 will be directed into the collection container 16. To assist in allowing the patient to remain calm and relaxed, the label 80 may be positioned on the outer surface of the collection container 16 so as to obstruct the patient's view of the blood sample 18 as it is directed into the collection container 16. Due to many patient's fear or uncomfortableness with the sight of blood, the position of the label 80 ensures the patient does not faint or become uncomfortable upon seeing the blood sample in the collection container 16. In one embodiment of the present disclosure, the label 80 is also positioned such that the user or physician has clear line of sight to the blood sample 18 being directed into the collection container 16. The label 80 may not obstruct the user or physician's line of sight of the interior of the collection container 16. In one example of the present disclosure, the label 80 may be positioned on an outer surface of the collection container 16 that is positioned underneath the holder 12.

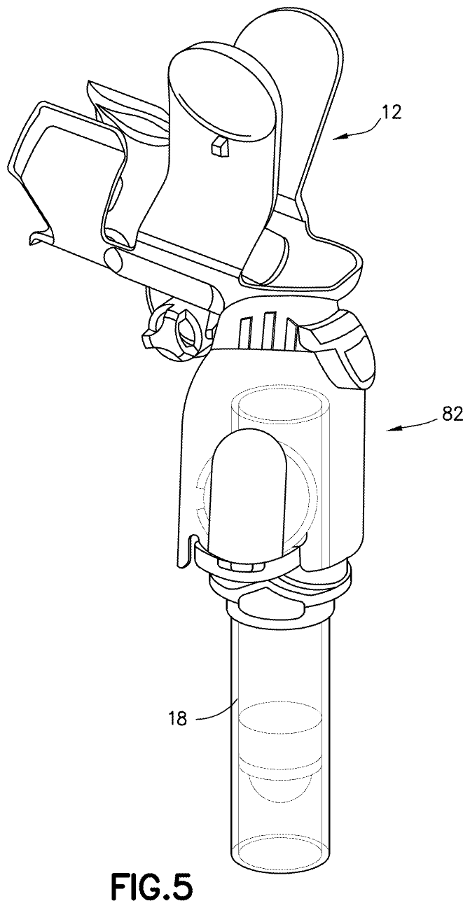

With reference to FIGS. 5-7, according to one embodiment of the present disclosure, the device 10 may also include a flash feature to immediately identify to a user or physician that a flow of blood has been obtained in the device 10. In one embodiment of the present disclosure, the device 10 may include a blood collector attachment 82 removably connected between the holder 12 and the collection container 16. A body of the blood collector attachment 82 may be made of a translucent material that allows a user or physician to view an internal cavity of the blood collector attachment 82 and the blood sample 18 therein. Furthermore, ribs provided on an inner surface of the blood collector attachment 82 may act as a light pipe or “flash” feature to receive a portion of the blood sample 18 to visibly present to the user or physician that the blood sample 18 is being directed into the collection container 16. The blood collector attachment 82 may be secured tightly to the holder 12 so the blood sample 18 is easily viewed.

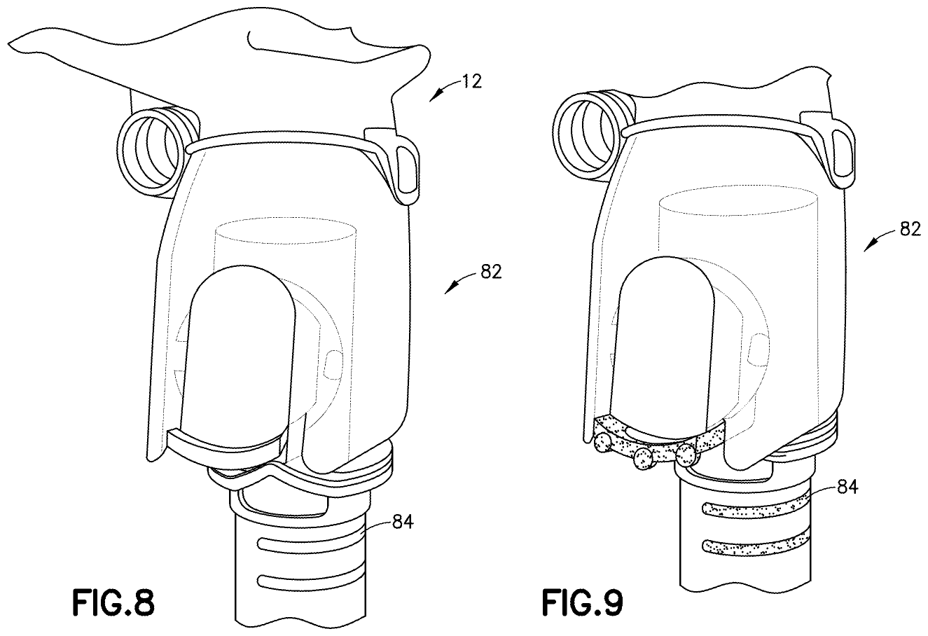

With reference to FIGS. 8 and 9, according to another embodiment of the present disclosure, the collection container 16 of the device 10 may include color-coded fill lines 84 to quickly and efficiently convey to the user or physician the type of collection container 16 that is being inserted into the blood collector attachment 82. As shown in FIG. 8, in one embodiment of the present disclosure, the collection container 16 may have fill lines 84 with a lighter shade of color. As shown in FIG. 9, in one embodiment of the present disclosure, the collection container 16 may have fill lines 84 that have a darker shade of color. In one embodiment of the present disclosure, the collection container 16 may include at least two fill lines 84 to assist the user or physician in determining the volume of blood collected in the collection container 16. The use of at least two fill lines 84 on the collection container 16 improves user experience and compliance when collecting a blood sample 18. The distance between the fill lines 84 and the thickness of the fill lines 84 may be adjusted as desired. Further, the location of the fill lines 84 on the collection container 16 may be adjusted to accommodate different desired volumes of blood in the collection container 16. Line thickness, color, and spacing for the fill lines 84 may be optimized for consistent fill experience to maintain sample quality and additive ratios. In one embodiment of the present disclosure, the user or physician may fill the collection container 16 to the top of the bottom fill line 84. Blood droplets from the patient might occur after the patient has reached the top of the bottom fill line 84 and the user or physician has stopped squeezing the wings 38 of the holder 12.

The foregoing features of the device 10 are directed to device usability improvements for hemophobia, trypanophobia, collection and color blindness.

While an embodiment of a capillary blood collection device is shown in the accompanying figures and described hereinabove in detail, other embodiments will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

The invention claimed is:

1. A device for obtaining a blood sample, the device comprising:
a holder for receiving a sample source, the holder having an actuation portion and a port;
a blood collector attachment removably connected to the holder; and
a collection container removably connectable to the blood collector attachment, the container defining a collection cavity, and the collection container comprising at least one fill line provided on a surface of the collection container.

2. The device of claim 1, wherein the at least one fill line is configured to indicate when a predetermined volume of the blood sample has been filled in the collection container.

3. The device of claim 1, wherein the collection container comprises two fill lines provided on the surface of the collection container.

4. The device of claim 1, wherein the at least one fill line of the collection container is color coded.

5. The device of claim 1, wherein the at least one fill line of the collection container has a lighter shade of color.

6. The device of claim 5, wherein the at least one fill line of the collection container has a darker shade of color.

7. A device for obtaining a blood sample, the device comprising:
a holder for receiving a sample source, the holder having an actuation portion and a port; and
a blood collector attachment removably connected to the holder, wherein the blood collector attachment includes a flash chamber to indicate when the blood sample has been drawn from a patient's finger held in the holder.

8. The device of claim 7, wherein the blood collector attachment is made of a material that is transparent such that a user of the device can visually identify the blood sample flowing through the blood collector attachment.

9. The device of claim 7, wherein the blood collector attachment includes a light rib on an inner surface thereof to visually indicate when the blood sample is moving through the blood collector attachment.

10. A device for obtaining a blood sample, the device comprising:
a holder for receiving a sample source, the holder having an actuation portion and a port; and
a lancet removably connected to the holder, wherein the lancet is positioned relative to the holder so as to obstruct a patient's view of the lancet removably connected to the holder.

11. The device of claim 10, wherein the port of the holder is configured to obstruct the patient's view of the lancet.

12. The device of claim 10, wherein, upon connection of the lancet into the holder, an audible clicking sound is generated to indicate that a positive connection between the lancet and the holder has been established.

13. A device for obtaining a blood sample, the device comprising:
a holder for receiving a sample source, the holder having an actuation portion and a port; and
a blood collector attachment removably connected to the holder; and
a collection container removably connected to the blood collector attachment, wherein a label positioned on the collection container is oriented so as to obstruct a patient's view of an inner cavity of the collection container.

14. The device of claim 13, wherein the label is positioned on the collection container at a location on the collection container underneath the holder.

15. The device of claim 13, wherein the label covers a portion of the collection container so the patient is unable to view the blood sample being directed into the collection container.
